(19) United States
(12) Patent Application Publication
(10) Pub. No.: US 2008/0319038 A1
(43) Pub. Date: Dec. 25, 2008

(54) PREVENTATIVE OR THERAPEUTIC AGENT FOR ACUTE RENAL FAILURE

(76) Inventors: Soroku Yagihashi, Hirosaki-shi (JP); Saori Ogasawara, Hirosaki-shi (JP); Chihiro Hibi, Nagoya-shi (JP); Noriaki Kato, Nagoya-shi (JP)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/139,787

(22) Filed: Jun. 16, 2008

Related U.S. Application Data
(63) Continuation-in-part of application No. PCT/JP2006/325054, filed on Dec. 15, 2006.

(30) Foreign Application Priority Data
Dec. 16, 2005 (JP) JP 2005-362579

(51) Int. Cl. A61K 31/4188; A61P 13/12

(52) U.S. Cl. 514/387

(57) ABSTRACT

An agent for prevention and treatment of acute renal failure comprising a hydantoin derivative represented by the general formula below as an active ingredient. One example of such compounds is (2S, 4S)-6-fluoro-2′,5′-dioxospiro[chroman-4,4′imidazolidine]-2-carboxamide.

### RELATED APPLICATIONS

This application is a continuation-in-part of and claims priority to PCT Application No. PCT/JP2006/325054, filed Dec. 15, 2006, and JP 2005-362579, filed Dec. 16, 2005 the disclosures of which are both hereby incorporated by reference.

### TECHNICAL FIELD

The present invention relates to novel medicinal use of hydantoin derivatives which are known as aldose reductase inhibitors (ARI).

### BACKGROUND ART

Systemic inflammatory response syndrome due to trauma, burns, pancreatitis, sepsis, or infection; disseminated intravascular coagulation syndrome; multiple organ failure; peripheral arterial occlusive disease; arteriosclerosis obliterans; and Crush syndrome are inflammatory diseases, and ischemic circulatory failure is initiated by the persistence or aggravation of inflammatory cytokinemia. As a result, vital organ damage and tissue injury occur, which evokes acute renal failure. That is, the above-mentioned disease is involved in vital prognosis, and currently, it significantly compromises patient's quality of life and is dreaded as a disease which shows a very high mortality rate. With reference to the treatment of the disease increased in severity, high dose steroid hormone therapy for the purpose of the inhibition of production of inflammatory cytokine and pharmacotherapy such as anticoagulant therapy for the purpose of the improvement of the systemic peripheral circulation have been performed. Alternatively, acute blood purification therapy, continuous renal support therapy, and plasma exchange for the purpose of the removal of causative substance have been performed. However, there is still no therapy to improve patient's prognosis and vital prognosis and the mortality rate is high.

Further, as a therapy for patients with end-terminal organ failure such as hepatic cirrhosis, organ transplantations such as liver transplantations have been performed around the world, but the postoperative management after the transplantation has been an important issue. In these circumstances, transplant results have been improved by various new immunosuppressive agents. However, for example, in liver transplantation, post-ischemia reperfusion after the transplantation may lead to acute renal failure. The frequency of hemodialysis is in the range of 2 to 21%, and it is said that the mortality rate in cases of dialysis is high.

From these reasons, there is an earnest desire for the advent of an effective and safe drug for the acute renal failure which is caused by the persistence or aggravation of systemic inflammatory response syndrome due to trauma, burns, pancreatitis, sepsis, or infection; disseminated intravascular coagulation syndrome; multiple organ failure; peripheral arterial occlusive disease; arteriosclerosis obliterans; and Crush syndrome. Further, in organ transplantation, there is an earnest desire for the advent of a drug to prevent and reduce the development of acute renal failure, which is a possible posttransplantation complication.

Regarding (2S,4S)-6-fluoro-2′,5′-dioxospiro[chroman-4,4′-imidazolidine]-2-carboxamide, its development for diabetic neuropathy is being studied as a compound which has potent aldose reductase inhibitory effects and high safety even when taken over a long period.

Regarding the hydantoin derivative including (2S,4S)-6-fluoro-2′,5′dioxospiro[chroman-4,4′-imidazolidine]-2-carboxamide, use in diabetic neuropathy is described in Japanese Patent Application Laid-Open (JP-A) No. 61-200991, use in circulation disease is described in JP-A No. 4-173791, use in various diseases accompanied with aging is described in JP-A No. 6-135968, use in diabetic simple retinopathy is described in JP-A No. 7-242547, use in diabetic keratopathy is described in JP-A No. 8-231549, use in diabetic maculopathy is described in WO2005/072066, and use in severe diabetic retinopathy is described in WO2005/079792. However, the effectiveness of the hydantoin derivatives for use of an agent for prevention and treatment of acute renal failure has not been reported.

(Patent document 1) JP-A No. 61-200991
(Patent document 2) JP-A No. 4-173791
(Patent document 3) JP-A No. 6-135968
(Patent document 4) JP-A No. 7-242547
(Patent document 5) JP-A No. 8-231549
(Patent document 6) WO20051072066
(Patent document 7) WO2005/079792

### DISCLOSURE OF THE INVENTION

### Problems to be solved by the Invention

The present invention was achieved in view of the above situations, and an objective of the present invention is to provide a preventive or therapeutic agent for acute renal failure. Particularly, the objective of the present invention is to provide an effective pharmaceutical agent for the acute renal failure which is caused by the persistence or aggravation of systemic inflammatory response syndrome due to particularly trauma, burns, pancreatitis, sepsis, or infection; disseminated intravascular coagulation syndrome; multiple organ failure; peripheral arterial occlusive disease; arteriosclerosis obliterans; and Crush syndrome; or posttransplantation complications.

### Means for Solving the Problems

First, the present inventors developed an experimental model of acute renal failure (hereinafter referred to as the present experimental model). In the present experimental model animal, creatine kinase (CK), urea nitrogen (BUN) and creatinine in the blood had increased significantly and the animal was presented with acute renal failure status which clinically requires renal dialysis. Subsequently, the present inventors evaluated (2S,4S)-6-fluoro-2′,5′-dioxospiro[chroman-4,4′-imidazolidine]-2-carboxamide (generic name: Fidarestat) in order to show the efficiency of hydantoin derivatives using the present experimental models. As a result, it was found that an increase of CK, BUN and creatinine in blood was reduced and the onset of acute renal failure was completely suppressed in the Fidarestat administered group (the ischemia reperfusion and Fidarestat administered group).

That is, the present invention is a preventive or therapeutic agent for acute renal failure which includes the hydantoin derivative represented by the following general formula as an active ingredient. A preferable example of the hydantoin derivative is (2S,4S)-6-fluoro-2′,5′-dioxospiro[chroman-4,4′-imidazolidine]-2-carboxamide.

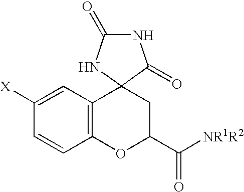

wherein X represents a halogen atom or a hydrogen atom, R1 and R2 independently represent a hydrogen atom or an optionally substituted C1 to C6 alkyl group, or R¹ and R², together with a nitrogen atom bound thereto, or optionally another nitrogen atom or an oxygen atom, are combined to form a 5- to 6-membered heterocycle. Here, the halogen atom of X is preferably a fluorine atom. Further, as a C1-6 alkyl group, a C1-3 alkyl group is preferable and a methyl group is particularly preferable.

Examples of acute renal failure include acute renal failures resulting from ischemia or ischemia reperfusion. That is, the acute renal failure which is caused by the persistence or aggravation of systemic inflammatory response syndrome due to particularly trauma, burns, pancreatitis, sepsis, or infection; disseminated intravascular coagulation syndrome; multiple organ failure; peripheral arterial occlusive disease; arteriosclerosis obliterans; and Crush syndrome; or posttransplantation complications can be exemplified.

### EFFECTS OF THE INVENTION

The present invention paves the way for drug therapy for the prevention or treatment of acute renal failure, particularly acute renal failure resulting from ischemia or ischemia reperfusion, namely, the acute renal failure which is caused by the persistence or aggravation of systemic inflammatory response syndrome due to particularly trauma, burns, pancreatitis, sepsis, or infection; disseminated intravascular coagulation syndrome; multiple organ failure; peripheral arterial occlusive disease; arteriosclerosis obliterans; and Crush syndrome; or posttransplantation complications. Further, the present invention causes no problems from a safety standpoint and provides a therapeutic agent which can be administered for a long-term period.

### BEST MODE FOR CARRYING OUT THE INVENTION

The present invention will be explained in more detail below.

The hydantoin derivative can be orally administered for example, as tablets, capsules, powders, granules, liquids or syrups, or can be parenterally administered as injectables, infusions or suppositories, which were formed by conventional pharmaceutical manufacturing techniques. For pharmaceutical manufacturing, in the case of solid formulations, pharmaceutically acceptable excipients such as starch, lactose, purified white sugar, glucose, crystalline cellulose, carboxycellulose, carboxymethylcellulose, carboxyethylcellolose, calcium phosphate, magnesium stearate, gum arabic and the like can be used and, if necessary, lubricants, binders, disintegrating agents, coating agents, coloring agents and the like can be incorporated. In addition, in the case of liquid formulations, stabilizers, solubilizers, suspending agents, emulsifiers, buffers, preservatives and the like can be used. The dose is different depending on symptoms, age, administration methods, dosage forms and the like and, in the normal case, it is preferable that the compound described above is administered to an adult in a range of 0.5 to 300 mg, preferably 1 to 150 mg per day in terms of the present compound for consecutive days, once or a few times a day.

The above-described formulations of the present invention are administered as a preventive or therapeutic agent for acute renal failure. Pathological conditions responsible for acute renal failure include systemic inflammatory response syndrome caused by trauma, systemic inflammatory response syndrome caused by burns, systemic inflammatory response syndrome caused by pancreatitis, systemic inflammatory response syndrome caused by sepsis, systemic inflammatory response syndrome caused by infection, disseminated intravascular coagulation syndrome, multiple organ failure, peripheral arterial occlusive disease, arteriosclerosis obliterans, Crush syndrome, or posttransplantation complications. The drug product of the present invention is especially effective for the acute renal failure which is caused by the persistence or aggravation of these conditions.

### EXAMPLES

### Test Example 1

### Effect of Fidarestat Administration on Renal Damage in Ischemia Reperfision Mice

1. Material and methods of pharmacological test

Sixteen- to twenty-week-old male C57BL/6 mice (Wild) were used in the experiment. The mice were divided into 4 groups, which were designated as sham-operation group (normal control group), ischemia operation group (ischemia reperfusion control group), ischemia operation+Fidarestat 40 mg/kg administration group (ischemia reperfusion/Fidarestat 40 mg/kg administration group), and ischemia operation+Fidarestat 150 mg/kg administration group (ischemia reperfusion/Fidarestat 150 mg/kg administration group), respectively. 40 or 150 mg/kg/day of Fidarestat was mixed in their diet and administered to the ischemia operation+Fidarestat administration groups 7 days before ischemia operation.

Plantar blood flow in these mice was measured with a laser blood flow meter under isoflurane anesthesia, and then the abdomen was incised. The abdominal aorta was exposed under a stereoscopic microscope and clipping was carried out at the portion distal to the renal artery bifurcation. Further, the right common iliac artery was also clipped and the right femoral artery was finally clipped. Thereafter, the abdomen was closed. The time of ischemia onset was set to the time when the femoral artery was clipped. From the time of ischemia onset, plantar blood flow was measured with a laser blood flow meter and the ischemia was confirmed. The clipping was released in order of the right femoral artery, right common iliac artery, and abdominal aorta in 3 hours from the ischemia onset and reperfusion was performed. At the time, the reperfusion status was confirmed with the laser blood flow meter. The reperfusion time was set to the time when the clipping of the aorta was released. The reperfusion was performed for 24 hours and the general condition was observed. Thereafter, heart blood was collected from the right atrium under isoflurane anesthesia. The collected heart blood was mixed with heparin and centrifuged at 3000 g. Then, the supernatant was collected therefrom, which was frozen for preservation at −80 degrees C. to use in a serologic test for CK, BUN, creatinine, and the like.

### 2. Results

Paralysis was observed in the ischemia reperfusion mice to which drug was not administered (ischemia reperfusion control group). The levels of creatine kinase (CK), urea nitrogen (BUN) and creatinine in blood were significantly increased compared with the normal control group and the mice presented with acute renal failure status which clinically requires renal dialysis. On the other hand, in the ischemia reperfusion/Fidarestat 40 mg/kg administration group and the ischemia reperfusion/Fidarestat 150 mg/kg administration group, the increase of CK in blood was reduced. Further, the increase of BUN and creatinine in blood was reduced to the level of the normal control group, and thus the onset of acute renal failure was completely suppressed.

TABLE 1
Average ± SEM, n = 4

| | CK | BUN | Creatinine |
|---|---|---|---|
| | (IU/L) | (mg/dl) | (mg/dl) |
| Normal control group | 330 ± 100 | 26 ± 2 | 0.13 ± 0.02 |
| Ischemia reperfusion control group | 187615 ± 77324 | 120 ± 33 | 0.44 ± 0.07 |
| Ischemia reperfusion/Fidarestat 40 mg/kg | 6978 ± 4904 | 40 ± 11 | 0.12 ± 0.01 |
| administration group | | | |
| Isehemia reperfusion/Fidarestat 150 mg/kg | 2348 ± 530 | 31 ± 1 | 0.10 ± 0.01 |
| administration group | | | |

### Test Example 2

### Effect of Fidarestat Administration on Renal Damage in Mice with Lipopolysaccharide (LPS)—Induced Inflammation

1. Material and methods of pharmacological test

Eight-week-old male CD-1 mice were used in the experiment. The mice were divided into 3 groups of normal control group (drug and LPS non-administration group), LPS control group (drug non-administration/LPS administration group), and LPS/Fidarestat administration group (Fidarestat and LPS administration group).

First, 10 mg/kg of Fidarestat (which was prepared with a solubilizing agent solution) was administered into the caudal vein of the Fidarestat administration group and the solubilizing agent solution was administered into the caudal vein of the drug non-administration group. After 5 minutes, Lipopolysaccharides (LPS: Escherichia coli; 0111: B4, Sigma) was prepared with a physiological salt solution, which was administered to the LPS administration group intraperitoneally at a concentration of 2 mg/kg and a physiological salt solution was administered to the LPS non-administration group intraperitoneally. After 24 hours, each of the groups of mice was anesthetized by ether inhalation and blood was collected from the abdominal vein using a heparinized syringe. Then, centrifugation was carried out under the following conditions: at 4 degree C., at 1,000×g, for 10 min. Plasma was then obtained. Blood urea nitrogen (BUN) and creatinine were measured using an autoanalyzer (Hitachi). In this regard, as a solubilizing agent, NMDG (N-methyl-D-glucamine) was used.

### 2. Results

As shown in Table 2, the mice of the LPS control group had higher levels of BUN and creatinine compared with those of the normal control group. On the other hand, low levels of BUN and creatinine were observed in mice of the LPS/Fidarestat administration group. It is assumed that the mouse with LPS-induced inflammation exhibits pathological conditions clinically similar to renal damage which is caused by the aggravation of systemic inflammatory response due to sepsis, and it was shown that Fidarestat was effective for renal function impairment in the model with LPS-induced inflammation.

TABLE 2
Average ± SEM

| | BUN | Creatinine |
|---|---|---|
| | (mg/kg) | (mg/dl) |
| Normal control group (n = 9) | 24.4 ± 1.1 | 0.048 ± 0.004 |
| LPS control group (n = 10) | 32.0 ± 6.0 | 0.076 ± 0.004 |
| LPS/Fidarestat administration group (n = 7) | 24.3 ± 2.2 | 0.067 ± 0.003 |

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

1. A method for preventing or treating acute renal failure in a mammal comprising administering to the mammal an effective amount of a compound represented by the formula: 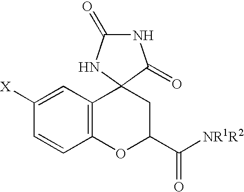
wherein X is a halogen atom or a hydrogen atom, R1 and R2 are independently a hydrogen atom, a C1 to C6 alkyl group or a substituted C1 to C6 alkyl group.

2. The method for preventing or treating acute renal failure according to claim 1, wherein the acute renal failure is resulting from ischemia or ischemia reperfusion.

3. The method for preventing or treating acute renal failure according to claim 1, wherein the acute renal failure is caused by the persistence or aggravation of a condition selected from the group consisting of systemic inflammatory response syndrome due to trauma, burns, pancreatitis, sepsis, infection, disseminated intravascular coagulation syndrome, multiple organ failure, peripheral arterial occlusive disease, arteriosclerosis obliterans, Crush syndrome and posttransplantation complications.

4. The method for preventing or treating acute renal failure according to claim 1, wherein X is fluorine.

5. The method for preventing or treating acute renal failure according to claim 1, wherein R1 and R2 are independently C1 to C3 alkyl groups.

6. The method for preventing or treating acute renal failure according to claim 1, wherein at least one of R1 and R2 is a methyl group.

7. The method for preventing or treating acute renal failure according to claim 1, wherein the compound is (2S,4S)-6-fluoro-2′,5′-dioxospiro[chroman-4,4′-imidazolidine]-2-carboxamide.
